(19) United States
(12) Patent Application Publication
(10) Pub. No.: US 2024/0423774 A1
(43) Pub. Date: Dec. 26, 2024

(54) TRANSFER INSTRUMENT

(71) Applicant: Terumo Kabushiki Kaisha, Tokyo (JP)

(72) Inventor: Koichi HAYAKAWA, Kanagawa (JP)

(73) Assignee: Terumo Kabushiki Kaisha, Tokyo (JP)

(21) Appl. No.: 18/740,742

(22) Filed: Jun. 12, 2024

(30) Foreign Application Priority Data
Jun. 20, 2023 (JP) 2023-100690

(51) Int. Cl. A61F 2/00

(52) U.S. Cl. CPC A61F 2/0063; A61F 2002/0072

(57) ABSTRACT

A first carrier member of a transfer instrument includes a first shaft disposed inside an outer cylinder so as to be movable along an axial direction, and a first support portion having a sheet shape and disposed at a distal end of the first shaft. The first support portion includes a first support surface capable of holding a medical sheet, a pair of first protrusions protruding upward from the first support surface, and a first bent portion provided at a boundary between the first support surface and each of the first protrusions and obtained by bending the first protrusion. The first support portion includes a first slit provided in each of the first bent portions and passing through the first support portion in a thickness direction.

### CROSS-REFERENCES TO RELATED APPLICATIONS

This application is based on and claims priority to Japanese Patent Application No. 2023-100690 filed on Jun. 20, 2023, the entire content of which is incorporated herein by reference.

### TECHNOLOGICAL FIELD

The present invention generally relates to a transfer instrument for transferring a medical sheet to a treatment site of a living body.

### BACKGROUND DISCUSSION

Japanese Patent Application Publication No. 2009-000511 (JP 2009-000511 A) discloses a transfer instrument for transferring a medical sheet (cell sheet) for use in, for example, organ transplantation to a treatment site of a living body. The transfer instrument includes an outer cylinder, a slide member slidably supported in the outer cylinder, and a seat support member provided at a distal end of the slide member.

### SUMMARY

It is desired to transfer a medical sheet to a treatment site more efficiently.

(1) A transfer instrument used to transfer a medical sheet to a treatment site of a living body includes an outer cylinder including a distal-end opening, and a carrier member including a shaft and a support portion. The shaft extends in an extending direction and the support portion is positioned at a distal end of the shaft so that the support portion and the shaft move together. The shaft is disposed in the outer cylinder, extends in an axial direction of the outer cylinder and is movable relative to the outer cylinder in the axial direction. The support portion has a sheet shape and includes a support surface configured to hold the medical sheet. The support portion has a width in a direction orthogonal to an extending direction in which the shaft extends, with the support surface having two sides spaced apart from one another in a width direction that is orthogonal to the extending direction. A pair of protrusions protrude upward from the sides of the support surface, a slit passes through the support portion in a thickness direction of the support portion, and the width of the support portion is larger than an inner circumferential length of the outer cylinder. The shaft is movable in a proximal direction relative to the outer cylinder when the shaft is positioned in the outer cylinder and the support portion is protruding distally beyond the distal-end opening of the outer cylinder with the medical sheet overlying the support surface of the support portion to retract the support portion into the outer cylinder and cause the support portion to deform into a curved shape and cause both sides of the support portion to protrude toward the support surface. The shaft is also movable in a distal direction relative to the outer cylinder when the support portion is retracted into the outer cylinder to cause the support portion to protrude distally beyond the distal-end opening of the outer cylinder.

With this transfer instrument, when the medical sheet is placed on the support surface of the support portion, moisture on the support surface can be effectively discharged to the outside of the support portion through the slit, so that, when the medical sheet placed on the support surface is transferred to the treatment site, it is possible to place the medical sheet at a predetermined position of the treatment site while reducing the influence of the moisture. It is therefore possible to efficiently transfer the medical sheet to the treatment site with the support portion. When the support portion is unfolded at the second position, the slit can effectively maintain the shaping (bending) of the protrusions relative to the support surface.

(2) The transfer instrument according to the above (1), in which the support portion may include a bent portion provided at a boundary between the support surface and each of the protrusions and obtained by bending the protrusion relative to the support surface, and the slit may be provided in the bent portion.

With this configuration, when the support portion is unfolded at the second position, the slit can effectively maintain the shaping (bending) of each of the protrusions with the bent portion as a base point.

(3) The transfer instrument according to the above (2), in which the slit may be provided along an extending direction of the bent portion.

With this configuration, the slit extends along the bent portion, so that the moisture on the support surface can be more effectively discharged to the outside of the support portion through the long slit, and the shaping of the protrusion can be more effectively maintained.

(4) The transfer instrument according to the above (2) or (3), in which the slit may include a plurality of cutouts spaced apart from each other in the extending direction of the bent portion.

With this configuration, a plurality of slits can be arranged in a wider range in the extending direction of the bent portion as compared with a structure including a single slit continuous along the extending direction of the bent portion. It is therefore possible to increase a discharge range of the moisture on the support surface and enable the shaping of each of the protrusions in a wider range.

(5) The transfer instrument according to any one of the above (2) to (4), in which the slit provided in each of the bent portions may include a single cutout extending along an extending direction of the bent portion. With this configuration, it is easy to form the slit in the support portion.

(6) The transfer instrument according to any one of the above (2) to (5), in which the support portion may include a proximal-end support portion serving as a proximal end side of the support portion, the proximal-end support portion may include a second bent portion, and the second bent portion may be provided with a second slit passing through the support portion in the thickness direction.

With this configuration, when the support portion is retracted in the outer cylinder from the proximal-end support portion, the support portion can be effectively deformed into a curved shape with the second bent portions as a base point, and the support portion can be smoothly retracted in the outer cylinder. The second slit allows the moisture on the support surface to be effectively discharged to the outside of the support portion.

(7) The transfer instrument according to the above (1), in which the slit may be provided in each of the protrusions.

With this configuration, it is possible to effectively shape the protrusions while discharging the moisture from the support portion to the outside through the slit.

The support portion of the transfer instrument includes the slit in the bent portion obtained by bending the protrusion relative to the support surface, and the slit passes through the support portion in the thickness direction. It is therefore possible to effectively discharge, when the medical sheet is placed on the support surface of the support portion, the moisture on the support surface to the outside of the support portion through the slit. It is therefore possible to place, when the medical sheet placed on the support surface is transferred to the treatment site, the medical sheet at a predetermined position of the treatment site while reducing the influence of the moisture. As a result, the medical sheet can be efficiently transferred to the treatment site with the support portion. When the support portion is unfolded at the second position, the slit can effectively maintain the shaping (bending) of the protrusions relative to the support surface.

According to another aspect, a transfer instrument for transferring a medical sheet to a treatment site of a living body comprises an outer cylinder having a through hole that extends from a distal-end opening at a distal end of the outer cylinder to a proximal-end opening at a proximal end of the outer cylinder, and a carrier member that includes a shaft and a support portion. The shaft has a distal end portion and the support portion has a proximal-end support portion and a distal-end support portion. The proximal-end support portion terminates at a connection joint that is fixed to the distal end portion of the shaft so that movement of the shaft results in movement of the support portion. The shaft is positionable in the through hole in the outer cylinder and is axially movable relative to the outer cylinder to move the support portion from a first position distal of the distal-end opening the outer cylinder to a second position retracted into the through hole in the outer cylinder by axially moving the shaft in a proximal direction relative to the outer cylinder, and from the second position to the first position by moving the shaft in a distal direction relative to the outer cylinder. The support portion includes a support surface configured to support the medical sheet and an oppositely facing surface, with the support portion including at least one slit that passes completely through the support portion so that the at least one slit opens to both the support surface of the support portion and the oppositely facing surface of the support portion so that moisture on the medical sheet supported on the support surface is able to flow through the at least one slit to be discharged to outside the support portion.

Another aspect involves a method comprising: moving a support portion toward a dish on which is supported a medical sheet, wherein the support portion is fixed to a distal end portion of a shaft and the shaft is positioned in an outer cylinder so that a proximal end of the shaft extends proximally beyond a proximal end of the outer cylinder and the support portion extends distally beyond a distal end of the outer cylinder. The support portion includes a support surface and an oppositely facing back surface, with the support portion including at least one slit that passes completely through the support portion so that the at least one slit opens to both the support surface of the support portion and the back surface of the support portion. The method also includes positioning the medical sheet on the support surface of the support portion so that the medical sheet is supported on the support portion while the support surface of the support portion faces the medical sheet, allowing moisture contained in the medical sheet to flow along the support surface and be discharged from the support surface toward the back surface of the support portion through the at least one slit, and axially moving the shaft and the support portion in a proximal direction relative to the outer cylinder to retract the support portion into the outer cylinder while the medical sheet is supported on the support portion.

### BRIEF DESCRIPTION OF THE DRAWINGS

FIG. 1 is a perspective view of a transfer instrument according to one embodiment.

FIG. 2 is an exploded perspective view of the transfer instrument illustrated in FIG. 1.

FIG. 3 is a plan view of a distal end of the transfer instrument illustrated in FIG. 1.

FIG. 4 is a longitudinal cross-sectional view taken along line IV-IV in FIG. 3.

FIG. 5 is a transverse cross-sectional view taken along line V-V in FIG. 3.

FIG. 6 is a plan view of a distal end of the transfer instrument having a slit in a first protrusion.

FIG. 7 is a plan view illustrating a first support portion in a flat state before a protrusion is formed.

FIG. 8 is a plan view of a distal end of a transfer instrument having a first support portion according to a first modification.

FIG. 9 is a flowchart illustrating a procedure of a transfer method for transferring a medical sheet using the transfer instrument illustrated in FIG. 1.

FIG. 10 is a first explanatory diagram of a sheet placing process.

FIG. 11 is a second explanatory diagram of the sheet placing process.

FIG. 12 is an explanatory diagram of a retracting process.

FIG. 13 is a cross-sectional view taken along line XIII-XIII in FIG. 12.

FIG. 14 is an explanatory diagram of a positioning process.

FIG. 15 is an explanatory diagram of an unfolding process.

FIG. 16 is an explanatory diagram of a moving process.

FIG. 17 is an explanatory diagram of a withdrawing process.

FIG. 18 is a plan view of a distal end of a transfer instrument having a first support portion according to a second modification.

### DETAILED DESCRIPTION

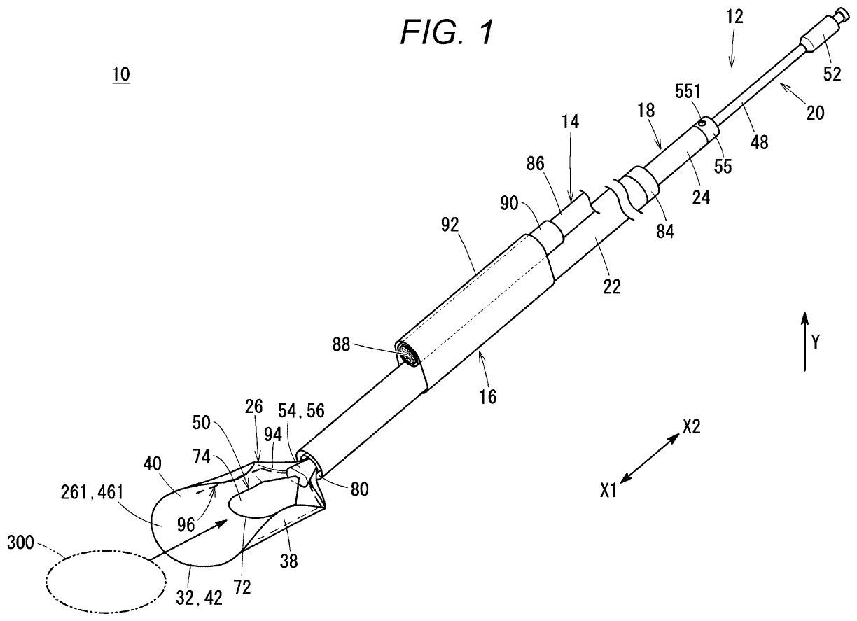

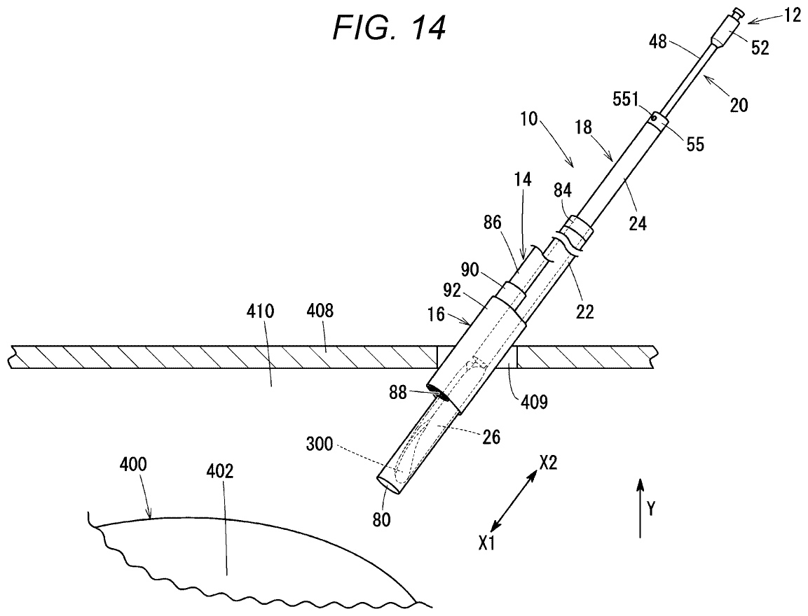

As illustrated in FIG. 1, a transfer instrument 10 according to the present embodiment is a medical instrument for transferring a medical sheet 300 to a treatment site of a living body. The transfer instrument 10 is used for, for example, the treatment of severe heart failure caused by ischemic heart disease. In this case, the medical sheet 300 is transplanted to a recipient site 402 of a heart 400 (the treatment site of the living body) (see FIGS. 14 to 17). The transfer instrument 10 is capable of attaching a plurality of the medical sheets 300 to the recipient site 402.

Examples of such a medical sheet 300 include pharmaceutical products or regenerative medicine products for medical use, a medical instrument, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 has a thickness of, for example, about 100 μm, and has a diameter of, for example, about 60 mm. The thickness and the diameter (size) of the medical sheet 300 are not limited these values and can be set as desired.

The medical sheet 300 may be a sheet to be transplanted to an organ (for example, lung, liver, pancreas, kidney, small intestine, esophagus, or the like) other than the heart 400. Further, the medical sheet 300 may be, for example, an anti-adhesion sheet as long as the sheet is for medical use.

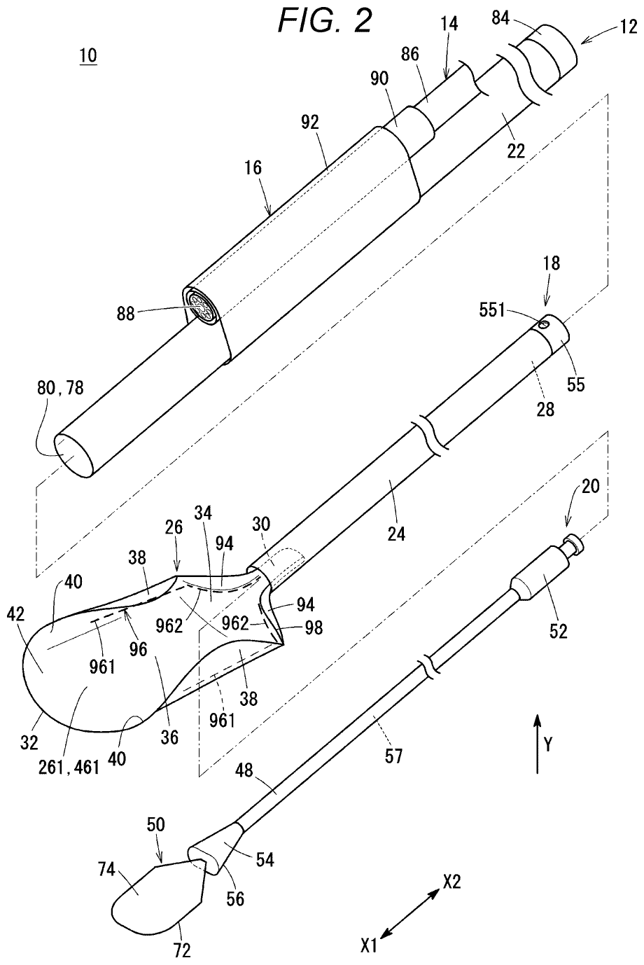

As illustrated in FIGS. 1 and 2, the transfer instrument 10 includes an instrument body 12, an endoscope 14, and a fixing member 16. The instrument body 12 includes a first carrier member 18, a second carrier member 20, and an outer cylinder 22. The transfer instrument 10 is not limited to a configuration including the endoscope 14.

In FIG. 2, the first carrier member 18 includes a first shaft 24 and a first support portion 26.

The first shaft 24 is a tubular body (in the present embodiment, a circular tube member) having a first lumen 28. The first lumen 28 opens at a distal end (end in a direction of arrow X1) of the first shaft 24 and opens at a proximal end (end in a direction of arrow X2) of the first shaft 24. The first shaft 24 has an airtight valve 55 provided at its proximal end with the valve 55 in close contact with an outer circumferential surface of the second shaft 48. The valve 55 has a marker 551 provided on its outer circumferential surface. When the transfer instrument 10 is used, the marker 551 is visible to the user. The first shaft 24 is not limited to a tubular body and may be a body other than a tubular body.

The first shaft 24 extends in an axial direction of the outer cylinder 22 and is disposed inside the outer cylinder 22 in a manner as to be movable along the axial direction. The first shaft 24 includes, for example, a resin material. Examples of the constituent material from which the first shaft 24 may be fabricated include, but are not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may include a metal material.

The first shaft 24 may be flexible. The first shaft 24 may have a flexible tube portion capable of maintaining a bent shape. In this case, the first shaft 24 can be bent into an any desired shape in a body cavity and can maintain the bent shape.

As illustrated in FIGS. 2 to 4, the first support portion 26 is attached to the distal end of the first shaft 24. The first support portion 26 includes or is fabricated from, for example, a resin material. The first support portion 26 can hold the medical sheet 300. A flexible resin sheet member (film member) is bent into a predetermined form to form the first support portion 26. Alternatively, the sheet member is shaped into a predetermined form by a sheet forming die to form the first support portion 26. It is preferable that the sheet member have, but not particularly limited to, a thickness of, for example, 100 μm or more and 200 μm or less. The first support portion 26 includes a first joint 30 and a first support body 32.

The constituent material from which the first support portion 26 may be fabricated preferably has transparency, and examples of the constituent material include, but are not particularly limited to, polyethylene, polycarbonate, polyamide, polystyrene, polypropylene, polyacetal resin, polyimide, polyetherimide, polyetheretherketone, polyethylene terephthalate, and fluororesin. Further, the first support portion 26 may have a mesh shape.

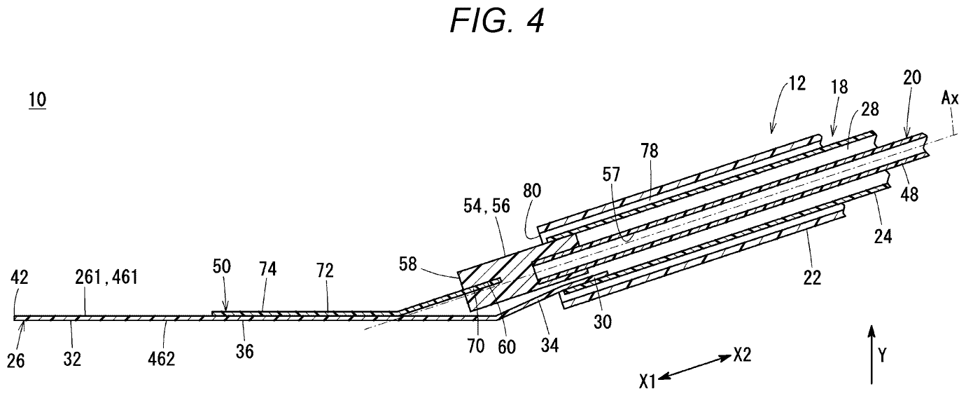

In FIG. 4, the first joint 30 is bonded to an inner circumferential surface of the distal end of the first shaft 24 with an adhesive. Examples of the adhesive include, but are not particularly limited to, a UV adhesive, a hot-melt adhesive, and an instant adhesive (for example, cyanoacrylate-based instant adhesive). The first joint 30 may be thermally fused to the inner circumferential surface of the first shaft 24. The first support portion 26 may be detachable from the distal end of the first shaft 24.

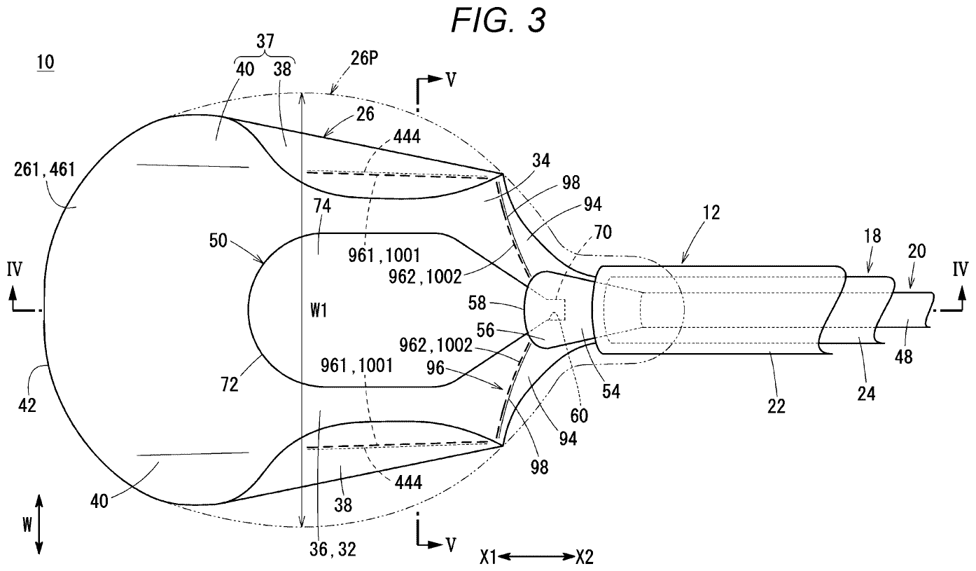

As illustrated in FIG. 2, the first support body 32 extends in a distal end direction from first joint 30. The first support body 32 includes a proximal-end support portion 34, an intermediate support portion 36, a pair of first protrusions 38, a pair of second protrusions 40, and a distal-end support portion 42. The first support body 32 includes a first support surface 261. The marker 551 of the first shaft 24 is disposed so as to face upward when the first support surface 261 faces upward. That is, the marker 551 allows the user to confirm the direction of the first support surface 261 of the first support portion 26. As shown in FIG. 3, the support portion 26 generally has a lengthwise extent between the distal-end support portion 42 (distal end portion) of the support portion 26 and the proximal-end support portion 34 (proximal end portion) of the support portion 26.

As illustrated in FIG. 3, the proximal-end support portion 34 is formed to be wider in its extending direction. In other words, both sides of the proximal-end support portion 34 in a width direction are tapered toward the first joint 30. The intermediate support portion 36 is formed in a tapered shape so as to be gradually narrower in width from the distal end toward the proximal end (in the direction of arrow X2).

The distal-end support portion 42 is connected to a distal end of the intermediate support portion 36 and distal ends of the pair of second protrusions 40. The distal-end support portion 42 protrudes in an arc shape in the distal end direction (the direction of arrow X1). That is, when viewed from a direction perpendicular to the first support surface 261 illustrated in FIG. 3, the distal-end support portion 42 of the first support portion 26 has an arc shape connecting the pair of second protrusions 40.

As illustrated in FIG. 4, the proximal-end support portion 34 extends roughly along an axis Ax of the first shaft 24 from a distal end of the first joint 30 in the distal end direction (the direction of arrow X1). The intermediate support portion 36 intersects with the axis Ax of the first shaft 24 and extends from a distal end of the proximal-end support portion 34 toward the distal end of the first support portion 26 (in the direction of arrow X1).

In FIGS. 2 and 3, the pair of first protrusions 38 protrude upward (in the direction of arrow Y) from both sides of the intermediate support portion 36 in the width direction orthogonal to a direction in which the first shaft 24 moves and inward in the width direction of the intermediate support portion 36. The pair of first protrusions 38 are connected to the proximal-end support portion 34. Each first protrusion 38 has a convex shape extending in a direction away from the first support surface 261.

As illustrated in FIG. 3, the pair of second protrusions 40 are connected to distal ends of the pair of first protrusions 38. The pair of second protrusions 40 protrude upward from both sides of the intermediate support portion 36 in the width direction and outward in the width direction of the intermediate support portion 36. Each second protrusion 40 is formed with a smaller curvature than the first protrusion 38. The second protrusions 40 are lower in protrusion height relative to the first support surface 261 than the first protrusions 38.

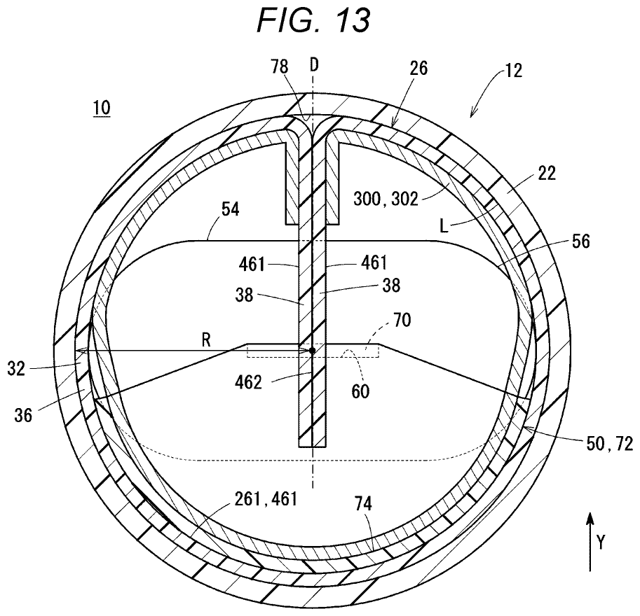

As illustrated in FIG. 3, a portion including both the first protrusion 38 and the second protrusion 40 of the first support portion 26 is hereinafter referred to as “protrusion 37”. Therefore, the protrusion 37 is provided as a pair on both sides of the first support portion 26 in the width direction. In FIG. 3, the direction (W direction) orthogonal to the axial direction of the first shaft 24 refers to the width direction of the first support portion 26. The W direction also refers to a width direction of other components (the outer cylinder 22 and the like). In the present embodiment, the first support portion 26 includes the pair of protrusions 37, and W1 in FIG. 3 denotes a width of the widest portion of the first support portion 26 when the first support portion 26 is unfolded to be flat. That is, the width W1 of the first support portion 26 is the maximum width of a first support portion 26P in a flat state before the pair of protrusions 37 are formed (before folded). The width W1 of the first support portion 26 is larger than an inner circumferential length L of the outer cylinder 22 (see FIG. 13). In FIG. 13, the inner circumferential length L of the outer cylinder 22 is a length of an inner circumferential surface along a circumferential direction of the outer cylinder 22 (L=2πR). R denotes the radius of the lumen 78 of the outer cylinder 22.

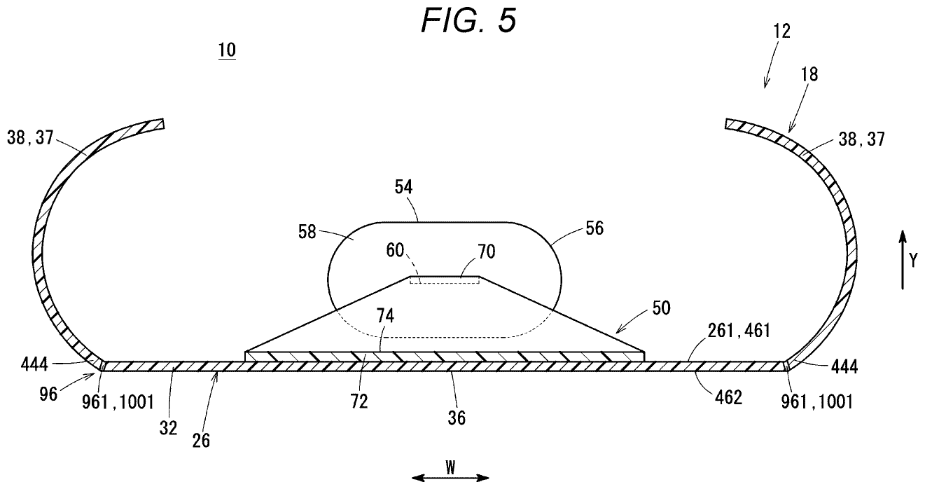

Each of the pair of first protrusions 38 has one of a pair of bent portions 444. Hereinafter, the bent portions 444 are each referred to as “first bent portion 444”. Each of the pair of first bent portions 444 causes a corresponding one of the pair of first protrusions 38 to bend relative to the first support surface 261 (intermediate support portion 36) of the first support portion 26 (see FIG. 5).

The first support body 32 includes a front surface 461 that faces upward (in the direction of arrow Y) and includes the first support surface 261, and a back surface 462 that is a surface opposite to the front surface 461. The first support surface 261 includes a flat surface continuous over an upper surface of the proximal-end support portion 34 and upper surfaces of the intermediate support portion 36 and the distal-end support portion 42. A lubricant may be applied to the first support surface 261 so as to allow a second support portion 50 (to be described later) of the second carrier member 20 to smoothly slide on the first support surface 261.

As illustrated in FIG. 2, the second carrier member 20 includes a second shaft 48, the second support portion 50, and a hub 52.

The second shaft 48 is a tubular body (in the present embodiment, a circular tube member) having a second lumen 57. The second shaft 48 is longer in the axial direction than the first shaft 24. The second shaft 48 is inserted into or positioned in the first lumen 28 of the first shaft 24 (see FIGS. 1 and 4). In other words, a distal end of the second shaft 48 protrudes in the distal end direction (the direction of arrow X1) relative to the distal-end opening of the first shaft 24. A proximal end of the second shaft 48 protrudes in the proximal end direction (the direction of arrow X2) relative to the proximal-end opening of the first shaft 24 (see FIG. 1). The second shaft 48 is provided extending along the first shaft 24 so as to be movable along the first shaft 24. The second shaft 48 is not limited to a tubular body and may be a body other than a tubular body.

The second shaft 48 is configured to follow the shape of the first support portion 26. As the constituent material from which the second shaft 48 may be fabricated, for example, a material more flexible than the constituent material of the first shaft 24 is selected. Specifically, examples of the constituent material of the second shaft 48 include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 4, the second shaft 48 includes a carrier holding portion 54 and a pressure-application portion 56 that is the distal end of the second shaft 48. The pressure-application portion 56 includes an elastic body such as an elastomer member. The pressure-application portion 56 presses the first support portion 26 against an inner surface of the outer cylinder 22 with the first support portion 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first support portion 26 in the distal end direction (the direction of arrow X1). In the present embodiment, the pressure-application portion 56 is provided with the carrier holding portion 54 that supports the second support portion 50. The carrier holding portion 54 includes the pressing surface 58 and an attachment hole 60.

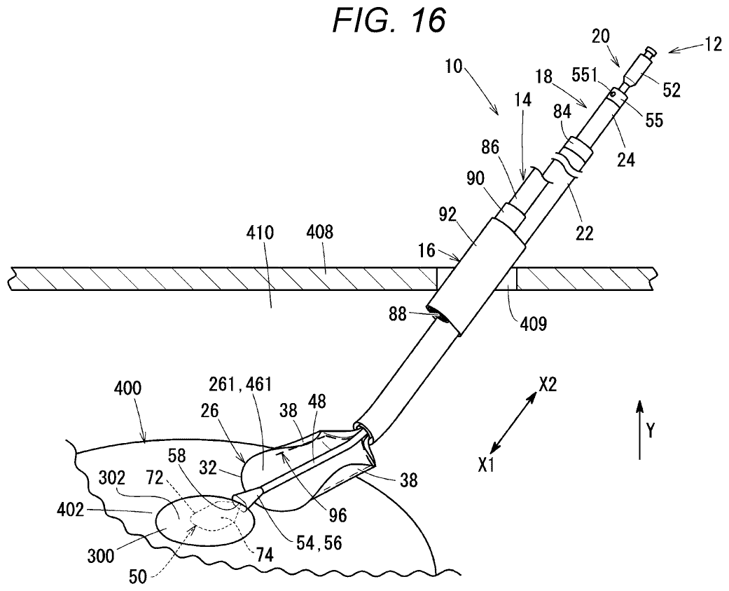

In FIG. 4, the pressing surface 58 is provided on a distal end surface of the carrier holding portion 54. The attachment hole 60 opens at the pressing surface 58. The second support portion 50 is attached to the pressing surface 58. The pressing surface 58 presses the outer edge surface of the medical sheet 300 in the distal end direction (the direction of arrow X1) (see FIG. 16).

In FIGS. 2 to 4, the second support portion 50 is a flexible sheet. The second support portion 50 includes a second joint 70 and a second support body 72. The second joint 70 is provided at a proximal end of the second support portion 50. The second joint 70 is provided at a proximal end of the second support body 72. The second joint 70 is inserted into or positioned in the attachment hole 60 of the carrier holding portion 54, and is bonded, for example. The second joint 70 may be joined to the attachment hole 60 of the carrier holding portion 54 by a suitable joining method other than adhesion. The second support portion 50 may be integrally formed with the carrier holding portion 54.

The second support body 72 extends in the distal end direction (the direction of arrow X1) from the second joint 70. The second support body 72 extending from the second joint 70 is shorter in the extending direction than the first support body 32 extending from the first joint 30. The second support body 72 is provided, on its upper surface, with a second support surface 74 on which the medical sheet 300 is placed. The second support surface 74 includes a flat surface. The second support body 72 is smaller than the first support body 32. That is, the second support surface 74 is smaller in area than the first support surface 261.

In FIG. 2, the hub 52 is attached to the proximal end of the second shaft 48.

In FIGS. 1 and 2, the outer cylinder 22 is a cylindrical member having a lumen 78. The lumen 78 has a distal-end opening 80 that opens at a distal end (end in the direction of arrow X1) of the outer cylinder 22. The lumen 78 opens at a proximal end (end in the direction of arrow X2) of the outer cylinder 22. The outer cylinder 22 is flexible. Examples of the constituent material from which the outer cylinder 22 may be fabricated are the same as the examples of the constituent material of the first shaft 24 described above. The first shaft 24 is inserted into or positioned in the lumen 78 of the outer cylinder 22. The outer cylinder 22 is shorter in length along the axial direction than the first shaft 24. The outer cylinder 22 is provided with, at its proximal end, an airtight valve 84 that is in close contact with an outer circumferential surface of the first shaft 24.

In FIGS. 2 and 4, a distal end surface of the outer cylinder 22 extends orthogonal to the axial direction of the outer cylinder 22.

As illustrated in FIG. 2, the endoscope 14 includes a long or elongated endoscope main body 86. A distal end of the endoscope main body 86 is fixed to an outer circumferential surface of the outer cylinder 22 by the fixing member 16 (see FIG. 1). An objective lens 88 provided on a distal end surface of the endoscope main body 86 is oriented toward the distal end of the outer cylinder 22 (in the direction of arrow X1). The distal end of the endoscope main body 86 is fixed to an intermediate portion of the outer cylinder 22 in the axial direction. The distal end of the endoscope main body 86 may be fixed to the distal end of the outer cylinder 22.

The fixing member 16 includes, for example, a fixing cylinder 90 and a fixing tube 92. The fixing cylinder 90 includes, for example, a hard resin material. The endoscope main body 86 can be inserted into or positioned in a lumen of the fixing cylinder 90. The fixing cylinder 90 is disposed along a longitudinal direction of the outer cylinder 22. The fixing tube 92 is a tube for fixing the fixing cylinder 90 at a predetermined position of the outer cylinder 22. The fixing tube 92 is, for example, a heat-shrink tube. The manner in which the distal end of the endoscope main body 86 is fixed to the outer cylinder 22 may be determined as desired.

As illustrated in FIG. 3, the first support body 32 of the first support portion 26 further includes a pair of proximal-end protrusions 94. The pair of proximal-end protrusions 94 are provided in the proximal-end support portion 34. The proximal-end protrusions 94 protrude obliquely upward from both sides of the proximal-end support portion 34 in the width direction (W direction) orthogonal to the direction in which the first shaft 24 moves, and protrude outward in the width direction of the proximal-end support portion 34 and obliquely in the proximal end direction. The pair of proximal-end protrusions 94 have their respective distal ends connected to the first protrusions 38. The pair of proximal-end protrusions 94 have their respective rear ends connected to the first joint 30 (see FIG. 2).

Each of the pair of proximal-end protrusions 94 has a corresponding one of a pair of bent portions 98. Hereinafter, the bent portions 98 are each referred to as “second bent portion 98”. Each of the pair of second bent portions 98 causes a corresponding one of the pair of proximal-end protrusions 94 to bend relative to the first support surface 261 of the first support portion 26. The second bent portions 98 are formed along the shape of the proximal-end support portion 34. As the first support portion 26 is viewed from above, the second bent portions 98 each have a roughly arc shape that is curved inward in the width direction of the first support portion 26 and is convexly curved in the distal end direction. In the width direction (W direction) of the first support portion 26, a separation distance between the pair of second bent portions 98 gradually decreases toward the first joint 30. That is, the pair of second bent portions 98 extend outward in the width direction from a proximal end of the proximal-end support portion 34 while extending in the distal end direction (the direction of arrow X1). The second bent portions 98 are each provided at a boundary between the first support surface 261 of the first support portion 26 and the corresponding proximal-end protrusion 94. The second bent portions 98 are each obtained by bending the corresponding proximal-end protrusion 94 relative to the first support surface 261.

As illustrated in FIG. 2, the first support portion 26 further includes a slit structure 96. As illustrated in FIG. 5, the slit structure 96 passes through the first support portion 26 in a thickness direction. As illustrated in FIG. 3, the slit structure 96 includes a slit 961 (hereinafter, referred to as “first slit 961”) provided in each of the first bent portions 444 and a slit 962 (hereinafter, referred to as “second slit 962”) provided in each of the second bent portions 98.

The first slit 961 is provided in each of the pair of first bent portions 444. Each of the first slits 961 is provided along an extending direction of the corresponding first bent portion 444. That is, the pair of first slits 961 are arranged at outer sides of the first support portion 26 in the width direction across the first support surface 261. The pair of first slits 961 are spaced apart from each other in the width direction (W direction) of the first support portion 26. The pair of first slits 961 are arranged roughly parallel to each other. The first bent portions 444 are each provided at a boundary between the first support surface 261 and the corresponding first protrusion 38 of the first support portion 26. The first bent portions 444 are each obtained by bending the corresponding first protrusion 38 relative to the first support surface 261.

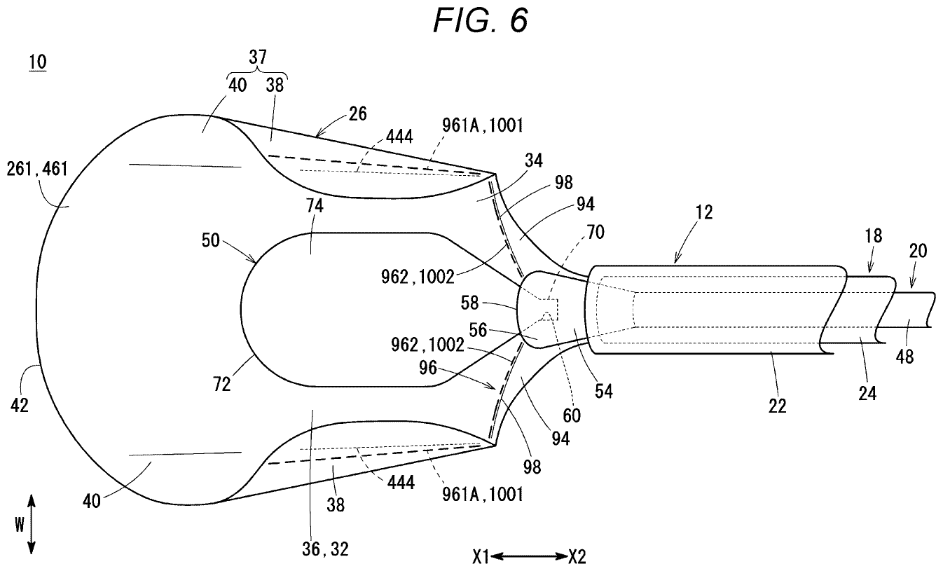

The first slits 961 are each a line including a plurality of first cutouts 1001. The first cutouts 1001 pass through the first support portion 26 in the thickness direction (see FIG. 5). Each of the first cutouts 1001 has a length along the extending direction of the first bent portions 444 larger than a width orthogonal to the extending direction of the first bent portions 444. The plurality of first cutouts 1001 are spaced apart from each other in the extending direction of the first bent portions 444. The number of first cutouts 1001 and the width of each first cutout 1001 orthogonal to the extending direction of the first bent portions 444 are set as desired. The first support surface 261 (front surface 461) of the first support portion 26 communicates with the back surface 462 of the first support portion 26 through the first slits 961. The location at which the first slits 961 are formed is not limited to the first bent portions 444. As illustrated in FIG. 6, for example, a first slit 961A may be formed in the pair of first protrusions 38.

Each of the first cutouts 1001 is not limited to the configuration where the length along the extending direction of the first bent portions 444 is larger than the width orthogonal to the extending direction of the first bent portions 444. For example, the first cutouts 1001 may have the length along the extending direction of the first bent portions 444 smaller than the width orthogonal to the extending direction of the first bent portions 444, or the width orthogonal to the extending direction of the first bent portions 444 and the length along the extending direction of the first bent portions 444 may be the same.

The second slits 962 are each provided in the corresponding second bent portion 98 of the first support portion 26. The second slits 962 are each provided along the extending direction of the second bent portions 98. As the first support portion 26 is viewed from above as illustrated in FIG. 3, the second slits 962 each have a roughly arc shape (i.e., are arc-shaped) that is convexly curved inward in the width direction of the first support portion 26. That is, the second slits 962 are provided in the proximal-end support portion 34 of the first support portion 26.

The second slits 962 are each a line including a plurality of second cutouts 1002. The second cutouts 1002 pass through the first support portion 26 in the thickness direction. A length along the extending direction of the second bent portions 98 is larger than a width orthogonal to the extending direction of the second bent portions 98. The plurality of second cutouts 1002 are spaced apart from each other in the extending direction of the second bent portions 98. The number of second cutouts 1002 and the width of each second cutout 1002 orthogonal to the extending direction of the second bent portions 98 are set as desired. The first support surface 261 (front surface 461) of the first support portion 26 communicates with the back surface 462 of the first support portion 26 through the second slits 962.

Each of the second cutouts 1002 is not limited to the configuration where the length along the extending direction of the second bent portions 98 is larger than the width orthogonal to the extending direction of the second bent portions 98. For example, the second cutouts 1002 may have the length along the extending direction of the second bent portions 98 smaller than the width orthogonal to the extending direction of the second bent portions 98, or the width orthogonal to the extending direction of the second bent portions 98 and the length along the extending direction of the second bent portions 98 may be the same.

As illustrated in FIG. 3, as the first support portion 26 is viewed from above, the slit structure 96 including the first and second slits 961 and 962 is disposed so as to surround the first support surface 261 of the first support portion 26. The slit structure 96 is not limited to the configuration including the first and second slits 961 and 962. For example, the slit structure 96 may include only the first slits 961.

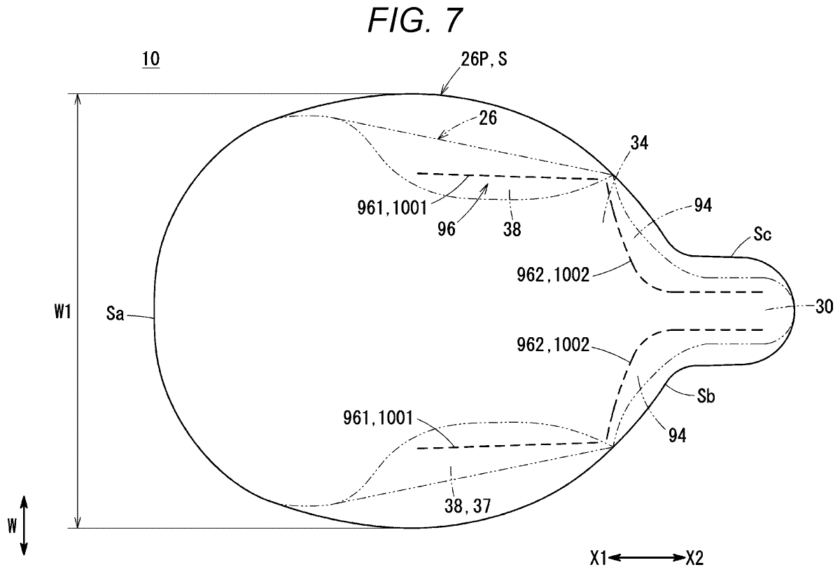

As illustrated in FIG. 7, the slit structure 96 is formed in advance in a first support portion 26P (hereinafter, also referred to as sheet member S) in a flat state before the pair of protrusions 37 are formed (before bent). The sheet member S has a roughly elliptical shape longer in the longitudinal direction. A distal end Sa of the sheet member S has a curved shape that is convexly curved in the distal end direction. A proximal end Sb of the sheet member S has a curved shape that is convexly curved in the proximal end direction. The proximal end Sb of the sheet member S has a bulging portion Sc that protrudes in the proximal end direction and serves as the first joint 30. The pair of first slits 961 of the slit structure 96 are arranged in an area extending in the proximal end direction (the direction of arrow X2) from a center of the sheet member S in the longitudinal direction. The first slits 961 are arranged at outer sides in the width direction from a width center so as to be spaced apart from each other and roughly parallel to each other. The first slits 961 are formed along the longitudinal direction of the sheet member S. The first slits 961 are each formed at a position to be the corresponding first bent portion 444 when both sides of the sheet member S in the width direction are bent to form the first protrusions 38. In other words, the first slits 961 (first cutouts 1001) are formed in advance at positions each serving as a boundary between the corresponding first protrusion 38 and the first support surface 261.

The pair of second slits 962 of the slit structure 96 are provided at the proximal end Sb of the sheet member S. The second slits 962 are each disposed in the proximal end direction relative to a proximal end of the corresponding first slit 961. The pair of second slits 962 are arranged so as to make their respective distal ends most separated from each other in the width direction and are curved from the distal ends toward the proximal ends to gradually come close (closer) to each other in the width direction. The proximal end of each second slit 962 extends to a middle of the bulging portion Sc. The proximal end of each second slit 962 may extend to a proximal end of the bulging portion Sc. The second slits 962 are each formed at a position to be the corresponding second bent portion 98 when the sheet member S is bent to form the proximal-end protrusions 94. In other words, the second slits 962 (second cutouts 1002) are formed in advance at positions each serving as a boundary between the corresponding proximal-end protrusion 94 and the first support surface 261.

When the sheet member S is shaped into a predetermined form by a sheet forming die (not illustrated) to form the first support portion 26, the first bent portions 444 having the first slits 961 are bent to form the first protrusions 38, and the second bent portions 98 having the second slits 962 are bent to form the proximal-end protrusions 94 (see FIG. 3).

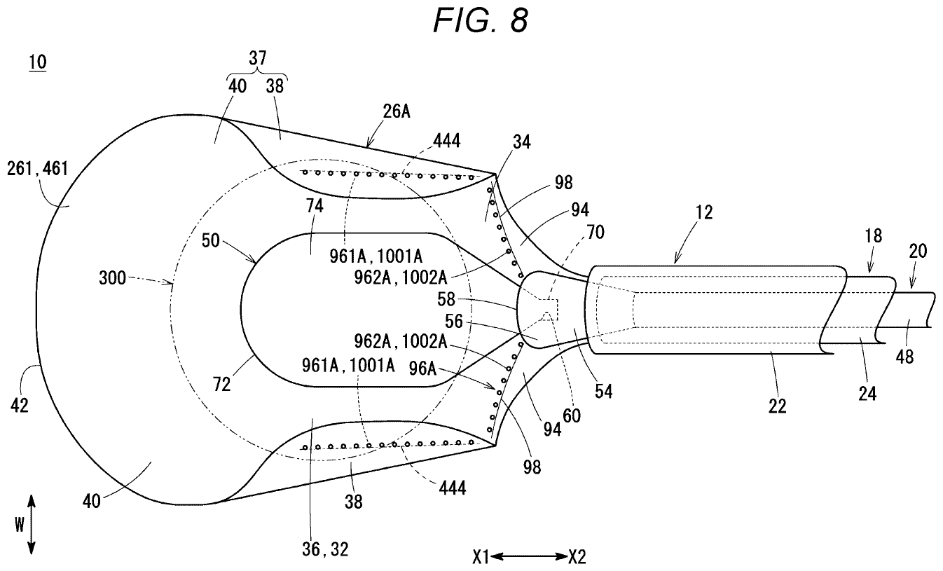

The first and second slits 961 and 962 are not limited to the configuration extending long (elongated) along the extending direction of the first and second bent portions 444 and 98. For example, as illustrated in FIG. 8, a first support portion 26A according to a first modification includes a slit structure 96A provided along the first bent portions 444 and the second bent portions 98. A slit 961A of the slit structure 96A includes a plurality of first cutouts 1001A each formed in or possessing a circular shape as viewed from above. Hereinafter, the slit 961A is referred to as “first slit 961A”. The plurality of the first cutouts 1001A (first slit 961A) pass through the first support portion 26A and are provided spaced apart from each other along the extending direction of the first bent portions 444. A slit 962A of the slit structure 96A includes a second cutout 1002A formed in a circular shape as viewed from above. Hereinafter, the slit 962A is referred to as “second slit 962A”. A plurality of the second cutouts 1002A (second slit 962A) pass through the first support portion 26A and are provided spaced apart from each other along the extending direction of the second bent portions 98. The shapes of the first cutouts 1001A and the second cutouts 1002A are not limited to a circular shape.

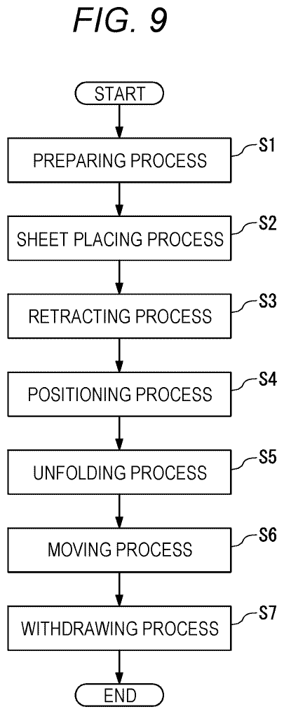

Next, a transfer method for transferring the medical sheet 300 to a treatment site of a living body will be described. Specifically, as illustrated in FIGS. 14 to 17, the transfer method for transferring the medical sheet 300 to the recipient site 402 of the heart 400 (the treatment site of the living body) during thoracoscopic surgery will be described. As illustrated in FIG. 9, the transfer method according to the present embodiment includes a preparing process, a sheet placing process, a retracting process, a positioning process, an unfolding process, a moving process, and a withdrawing process.

First, in the preparing process (step S1), the transfer instrument 10 according to the present embodiment described above is prepared. The following description will be given on the assumption that a state as illustrated in FIG. 1 is an initial state of the transfer instrument 10. In the initial state, the first shaft 24 and the second shaft 48 are moved in the distal end direction (the direction of arrow X1) relative to the outer cylinder 22 so as to be at a protruding position (second position) where the first support portion 26 and the second support portion 50 protrude from the distal-end opening 80 of the outer cylinder 22 in the distal end direction. The first and second support portions 26 and 50 are each unfolded when moving out of the outer cylinder 22 in the distal end direction, and the second support portion 50 is positioned on the first support surface 261 of the first support portion 26. That is, the second support portion 50 is positioned at a retracted position where the second support portion 50 is placed on the first support surface 261 of the first support portion 26. At this time, a proximal end of the carrier holding portion 54 is in the first lumen 28 of the first shaft 24.

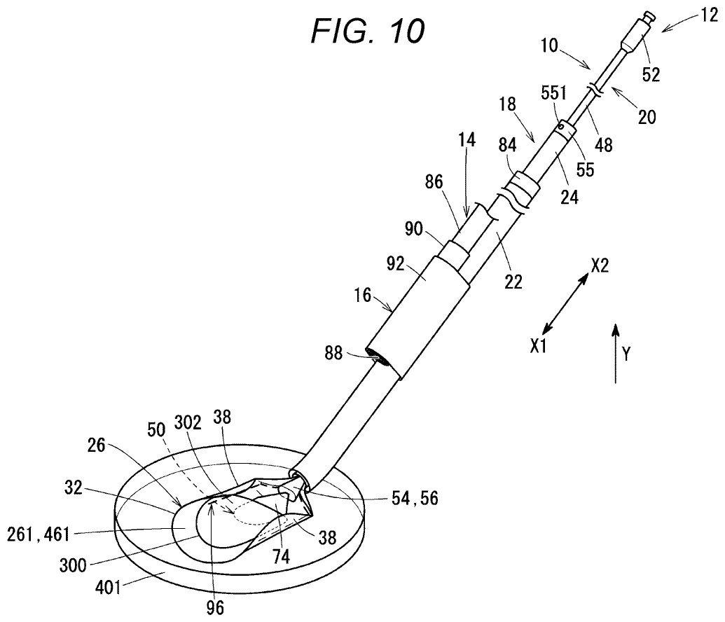

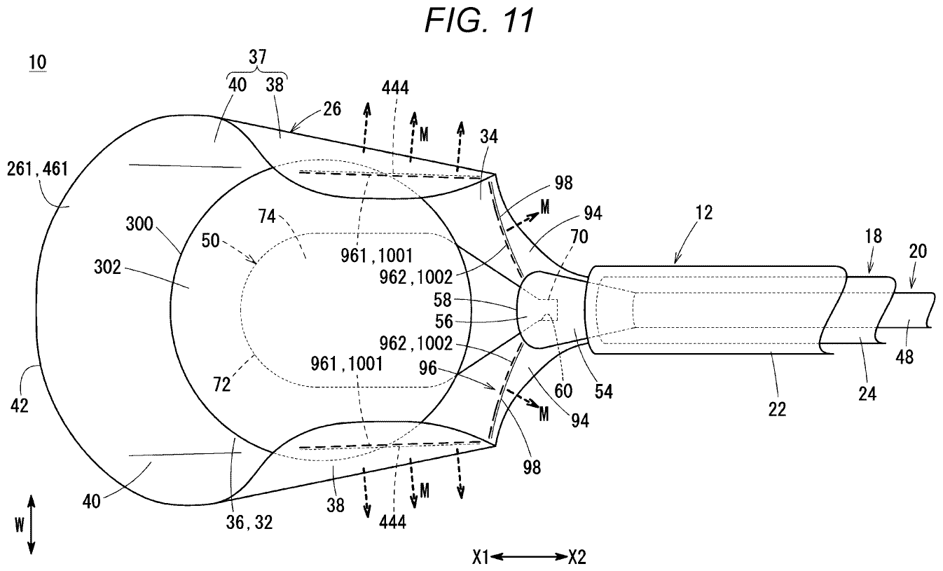

Next, in the sheet placing process (step S2), as illustrated in FIG. 10, the medical sheet 300 placed on or located in a Petri dish 401 is placed on the second support surface 74. The medical sheet 300 protrudes outward from the second support portion 50 with the medical sheet 300 placed on the second support surface 74. The first support surface 261 supports an overhanging portion 302 of the medical sheet 300 that sticks out over the second support portion 50. As illustrated in FIG. 11, at this time, in the first support portion 26, moisture M contained in the medical sheet 300 flows along the first support surface 261 and is discharged from the first support surface 261 toward the back surface 462 (to the outside) through the first slits 961 (the plurality of first cutouts 1001) and the second slits 962 (the plurality of second cutouts 1002) (see dashed arrows in the drawing).

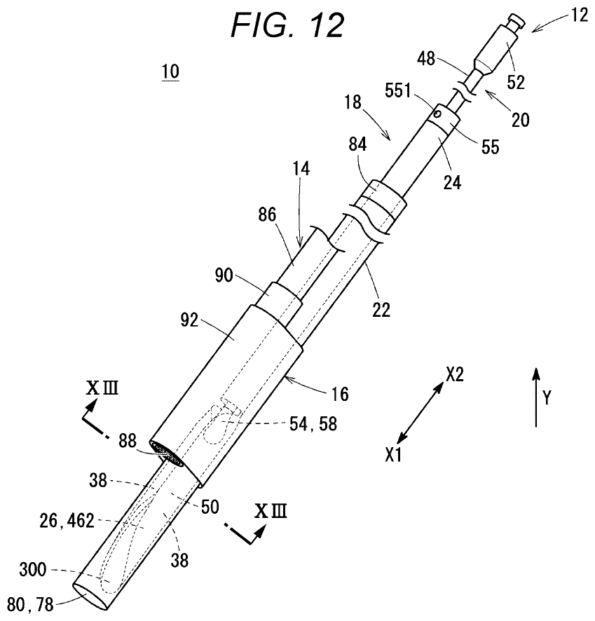

Subsequently, in the retracting process (step S3 in FIG. 9), as illustrated in FIG. 12, the medical sheet 300 is retracted in the outer cylinder 22 together with the first support portion 26 and the second support portion 50 at a retracted position (first position). Specifically, the first shaft 24 of the first carrier member 18 and the second shaft 48 of the second carrier member 20 are moved together in the proximal end direction (the direction of arrow X2) relative to the outer cylinder 22.

Then, the proximal-end support portion 34 (see FIG. 2) of the first support portion 26 is pulled in the proximal end direction through the distal-end opening 80 of the outer cylinder 22. At this time, when both the tapered sides of the proximal-end support portion 34 come into contact with the distal-end opening 80 of the outer cylinder 22, a force acts on the proximal-end support portion 34 to cause the proximal-end support portion 34 to curl along the circumferential direction of the outer cylinder 22. Therefore, the proximal-end support portion 34 is smoothly pulled into the outer cylinder 22 while curling. At this time, the first support portion 26 is retracted in the outer cylinder 22 while curling in a conical shape such that the distal end side of the first support portion 26 becomes larger in diameter than the proximal-end support portion 34.

When the proximal-end support portion 34 of the first support portion 26 becomes deformed, a force acts on the intermediate support portion 36 of the first support portion 26 to cause the intermediate support portion 36 to curl along the circumferential direction of the outer cylinder 22, so that the intermediate support portion 36 (see FIG. 2) is pulled into the outer cylinder 22 while curling. At this time, the intermediate support portion 36 becomes deformed into a cylindrical shape along the inner surface of the outer cylinder 22. Each of the pair of first protrusions 38 is curved such that the front surface 461 of the first support portion 26 faces inward and the back surface 462 of the first support portion 26 faces outward. As illustrated in FIG. 13, the back surfaces 462 of both sides of the first support portion 26 in the width direction come into contact with each other on an imaginary line D extending orthogonal to the center axis of the outer cylinder 22. One of the first protrusions 38 and the other first protrusion 38 come into contact with each other and are retracted downward (toward the first support surface 261, the front surface 461).

Accordingly, the back surface 462 of the first support portion 26 is curved and is in close contact with the inner surface of the outer cylinder 22, each of the first protrusions 38 is further curved to fold back toward the center of the outer cylinder 22, and a free end of each of the first protrusions 38 is positioned below the center axis of the outer cylinder 22. That is, the first support portion 26 is curved in a heart shape along the inner surface of the outer cylinder 22.

The heart shape refers to a roughly round shape including two convex shapes, a convex shape on one side and a convex shape on the other side. In a case where the heart shape is formed in the lumen 78 of the tubular body (outer cylinder 22), the two convex shapes protruding toward the opposite sides along the inner surface of the tubular body come close to each other so as to bring their respective circumferential surfaces into partial contact with each other, so that the entire contour becomes a roughly round shape along the inner surface of the tubular body (refer to the shape of the first support portion 26 in FIG. 13).

Along with the curved deformation of the first support portion 26, the second support portion 50 is similarly curved and deformed along the first support portion 26 inside the first support portion 26 (on the front surface 461 side). Along with the curved deformation of the first support portion 26 and the second support portion 50, the medical sheet 300 is deformed into a shape corresponding to the shape of the first support body 32 and the shape of the second support body 72, and the medical sheet 300 is retracted in the outer cylinder 22 accordingly.

As illustrated in FIG. 12, when the first support portion 26 is entirely inserted into or positioned in the outer cylinder 22, the retracting process is complete. At the completion of the retracting process, the first support portion 26 may partially protrude from the distal-end opening 80 of the outer cylinder 22. In this case, the state where the first support portion 26 partially protrudes from the distal-end opening 80 of the outer cylinder 22 corresponds to the first position of the first support portion 26.

Subsequently, in the positioning process (step S4 in FIG. 9), as illustrated in FIG. 14, the transfer instrument 10 is inserted into a chest cavity 410 through an incision 409 in a chest 408. At this time, the distal end of the transfer instrument 10 is positioned near the recipient site 402 of the heart 400, and the distal end of the endoscope 14 is positioned in the chest cavity 410. Before the transfer instrument 10 is inserted into the chest cavity 410, a liquid supply instrument (not illustrated) may be connected to a connection port of the hub 52 to introduce a liquid (for example, a physiological saline solution).

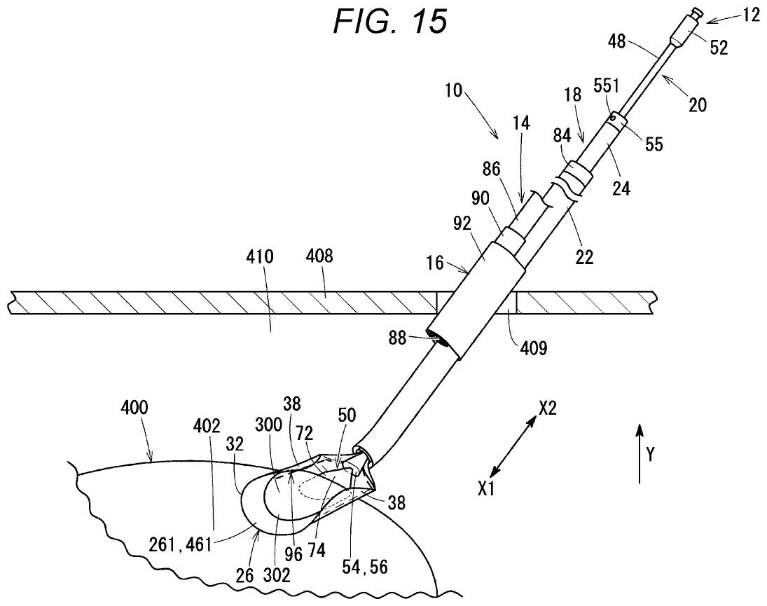

Subsequently, in the unfolding process (step S5 in FIG. 9), as illustrated in FIG. 15, the first support portion 26, the second support portion 50, and the medical sheet 300 are unfolded. Specifically, in the unfolding process, with the first shaft 24 gripped, the first shaft 24 is moved in the distal end direction (the direction of arrow X1) relative to the outer cylinder 22. Accordingly, the valve 55 of the first shaft 24 causes the second shaft 48 to move together with the first shaft 24 in the distal end direction (the direction of arrow X1). Then, the first support portion 26 that has moved out of the distal-end opening 80 of the outer cylinder 22 returns to the original shape due to its restoring force. At the second position where the first support portion 26 is unfolded, the second support portion 50 unrolls flat.

In the unfolding process, of the second carrier member 20, the second support surface 74 on which the medical sheet 300 is placed is entirely positioned on the first support surface 261. At this time, the medical sheet 300 is supported by the first support surface 261 and the second support surface 74. It is therefore possible to prevent the overhanging portion 302 of the medical sheet 300 from becoming crinkled before the medical sheet 300 is transferred to the recipient site 402 of the heart 400.

Next, in the moving process (step S6 in FIG. 9), as illustrated in FIG. 16, the second carrier member 20 is moved in the distal end direction (the direction of arrow X1) relative to the first carrier member 18, so that the second support portion 50 on which the medical sheet 300 is placed is moved from the retracted position to an advanced position, and the second support portion 50 protrudes in the distal end direction (the direction of arrow X1) relative to the distal end of the first support portion 26. Specifically, in the moving process, the second shaft 48 is moved in the distal end direction relative to the first shaft 24.

Accordingly, the second support portion 50 moves in the distal end direction (the direction of arrow X1) relative to the first support portion 26. At this time, when the distal end surface of the carrier holding portion 54 (pressure-application portion 56) presses the outer edge surface of the medical sheet 300 in the distal end direction, the medical sheet 300 is entirely positioned further than the first support portion 26 in the distal end direction. In this moving process, the medical sheet 300 is moved to above the recipient site 402 of the heart 400 to bring the overhanging portion 302 of the medical sheet 300 into contact with the recipient site 402.

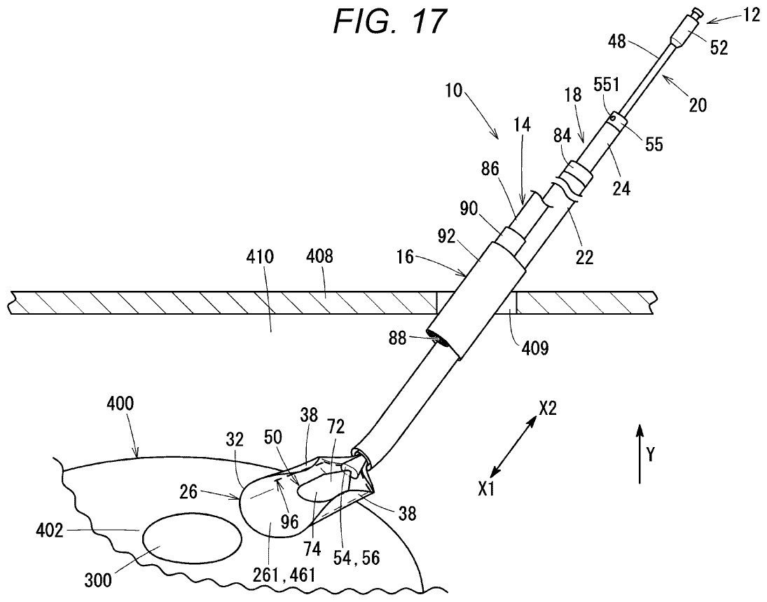

Subsequently, in the withdrawing process (step S7 in FIG. 9), as illustrated in FIG. 17, the second carrier member 20 is moved from the second position to the first position to withdraw the second support portion 50 from between the recipient site 402 and the medical sheet 300. Accordingly, the medical sheet 300 entirely comes into contact with the surface of the recipient site 402. This is the end of the transfer of the medical sheet 300 to the recipient site 402. Subsequently, the transfer instrument 10 is withdrawn from the chest 408 with the first support portion 26 and the second support portion 50 retracted in the outer cylinder 22.

The present embodiment has the following effects.

As illustrated in FIG. 3, the first support portion 26 of the first carrier member 18 of the transfer instrument 10 includes the first support surface 261 capable of holding the medical sheet 300, the pair of protrusions 37 protruding upward from both sides of the first support surface 261 in the width direction, and the first slits 961 passing through the first support portion 26 in the thickness direction.

With this configuration, as illustrated in FIG. 11, when the medical sheet 300 is placed on the first support portion 26, the moisture M on the first support surface 261 can be effectively discharged to the outside of the first support portion 26 through the first slits 961, so that, when the medical sheet 300 placed on the first support surface 261 is transferred to the recipient site 402 of the living body as illustrated in FIG. 16, it is possible to place the medical sheet 300 at a predetermined position of the recipient site 402 while reducing the influence of the moisture M. It is therefore possible to efficiently transfer the medical sheet 300 to the recipient site 402 with the first support portion 26. When the first support portion 26 is unfolded at the second position, the first slits 961 can effectively maintain the shaping (bending) of the first protrusions 38 relative to the first support surface 261.

As illustrated in FIG. 5, the first support portion 26 includes the first bent portions 444 provided at a boundary between the corresponding protrusion 37 and the first support surface 261 and obtained by bending the protrusion 37 relative to the first support surface 261, and the first slits 961 are each provided in the corresponding first bent portion 444. With this configuration, when the first support portion 26 is unfolded at the second position, the first slits 961 can effectively maintain the shaping (bending) of the first protrusions 38 with the first bent portions 444 as a base point.

As illustrated in FIG. 11, the first slits 961 are provided along the extending direction of the first bent portions 444. With this configuration, the moisture M on the first support surface 261 can be more effectively discharged to the outside of the first support portion 26 with the first slits 961 extending long along the first bent portions 444. The shaping of the first protrusions 38 relative to the first support surface 261 can be more effectively maintained.

As illustrated in FIG. 3, the first slits 961 each include the plurality of first cutouts 1001 spaced apart from each other in the extending direction of the first bent portions 444. With this configuration, as compared with a structure including a single slit continuous along the extending direction of a bent portion, there is no continuous cutout along the extending direction of the first bent portions 444 due to the presence of the plurality of first cutouts 1001. It is therefore possible to arrange, in the first support portion 26, the plurality of first cutouts 1001 in a wider range to increase a discharge range of the moisture M on the first support surface 261 and to enable the shaping of the first protrusion 38 in a wider range.

The first support portion 26 includes the proximal-end support portion 34 serving as the proximal end side of the first support portion 26, the proximal-end support portion 34 includes the second bent portions 98, and the second bent portions 98 are each provided with the second slit 962 passing through the first support portion 26 in the thickness direction.

With this configuration, when the first support portion 26 is retracted in the lumen 78 of the outer cylinder 22 from the proximal-end support portion 34, the first support portion 26 can be effectively deformed into a curved shape with the second bent portions 98 as a base point, and the first support portion 26 can be smoothly retracted in the lumen 78 of the outer cylinder 22. In other words, the second bent portions 98 including the second slits 962 can provide a trigger for (can facilitate) the first support portion 26 to undergo deformation into a curved shape. The second slits 962 allow the moisture M on the first support surface 261 to be effectively discharged to the outside of the first support portion 26.

As illustrated in FIG. 6, the first slits 961A are each provided in a corresponding one of the pair of first protrusions 38. With this configuration, it is possible to effectively shape the first protrusions 38 while discharging the moisture M from the first support portion 26 to the outside through the first slits 961A.

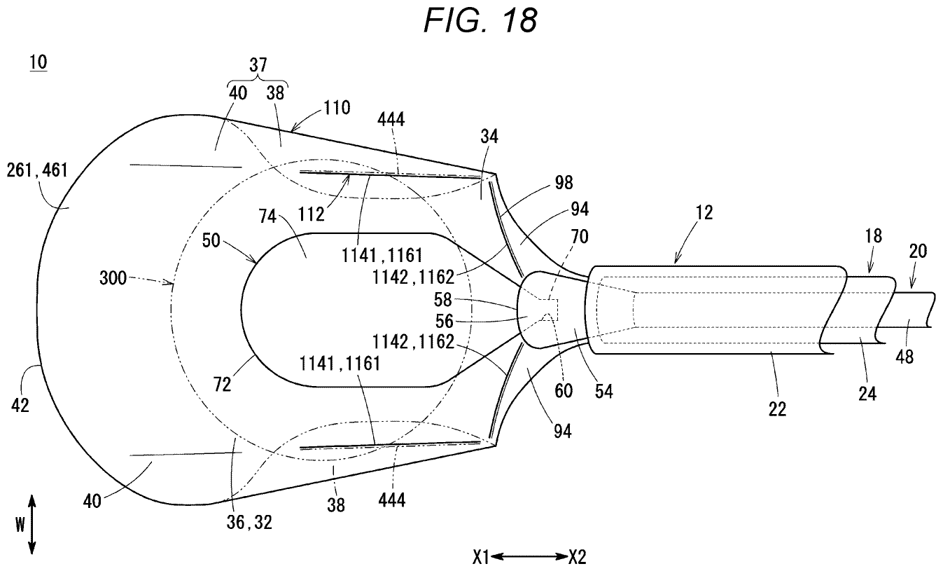

The first slits 961 are not limited to the configuration including the plurality of first cutouts 1001 in the extending direction of the first bent portions 444. The second slits 962 are not limited to the configuration including the plurality of second cutouts 1002 in the extending direction of the second bent portions 98. For example, as illustrated in FIG. 18, a first support portion 110 includes a slit structure 112. The slit structure 112 includes a pair of slits 1141 (hereinafter, referred to as “first slit 1141”) each provided in the corresponding first bent portion 444 and a pair of slits 1142 (hereinafter, referred to as “second slit 1142”) each provided in the corresponding second bent portion 98. The slit structure 112 is not limited to the configuration including the first and second slits 1141 and 1142. For example, the slit structure 112 may include only the first slits 1141.

Each of the first slits 1141 includes a long single first cutout 1161 extending along the extending direction of the first bent portions 444. Each of the second slits 1142 includes a single long second cutout 1162 extending along the extending direction of the second bent portions 98. Note that only either the first slit 1141 or the second slit 1142 may be a single continuous cutout.

The present modification has the following effects.

As illustrated in FIG. 18, in the slit structure 112 of the first support portion 110, the pair of first slits 1141 each include the single first cutout 1161 extending along the extending direction of the first bent portions 444, and the pair of second slits 1142 each include the single second cutout 1162 extending along the extending direction of the second bent portions 98. Therefore, the slit structure 112 can be easily formed as compared with a configuration where a slit structure including a plurality of cutouts is formed along the extending direction of the first and second bent portions 444 and 98. Through the single continuous first and second slits 1141 and 1142 (slit structure 112), the moisture M on the first support surface 261 can be effectively discharged to the outside of the first support portion 26.

The present invention is not limited to the above-described disclosure, and various configurations can be adopted without departing from the gist of the present invention. That is, the detailed description above describes embodiments of a transfer instrument and transfer method for transferring a medical sheet to a treatment site of a living body, representing examples of the new transfer instrument and transfer method disclosed here, but the invention is not limited to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A transfer instrument used to transfer a medical sheet to a treatment site of a living body, the transfer instrument comprising:
an outer cylinder including a distal-end opening;
a carrier member including a shaft and a support portion, the shaft extending in an extending direction and the support portion being positioned at a distal end of the shaft so that the support portion and the shaft move together, the shaft being positionable in the outer cylinder so that the shaft extends in an axial direction of the outer cylinder and is movable relative to the outer cylinder in the axial direction, the support portion having a sheet shape;
the support portion including
a support surface configured to hold the medical sheet, the support portion having a width in a direction orthogonal to an extending direction in which the shaft extends, the support surface having two sides spaced apart from one another in a width direction that is orthogonal to the extending direction;
a pair of protrusions protruding upward from the sides of the support surface;
a slit passing through the support portion in a thickness direction of the support portion;
the width of the support portion being larger than an inner circumferential length of the outer cylinder;
the shaft being movable in a proximal direction relative to the outer cylinder when the shaft is positioned in the outer cylinder and the support portion is protruding distally beyond the distal-end opening of the outer cylinder with the medical sheet overlying the support surface of the support portion to retract the support portion into the outer cylinder and cause the support portion to deform into a curved shape and cause both sides of the support portion to protrude toward the support surface; and
the shaft being movable in a distal direction relative to the outer cylinder when the support portion is retracted into the outer cylinder to cause the support portion to protrude distally beyond the distal-end opening of the outer cylinder.

2. The transfer instrument according to claim 1, wherein the support portion includes a bent portion provided at a boundary between the support surface and each of the protrusions and obtained by bending the protrusion relative to the support surface, and the slit being provided in the bent portion.

3. The transfer instrument according to claim 2, wherein the slit is provided along an extending direction of the bent portion.

4. The transfer instrument according to claim 3, wherein the slit includes a plurality of cutouts spaced apart from each other in the extending direction of the bent portion.

5. The transfer instrument according to claim 2, wherein the slit provided in each of the bent portions includes a single cutout extending along an extending direction of the bent portion.

6. The transfer instrument according to claim 1, wherein the support portion includes a proximal-end support portion serving as a proximal end side of the support portion, and
the proximal-end support portion includes a second bent portion, the second bent portion being provided with a second slit passing through the support portion in the thickness direction of the support portion.

7. The transfer instrument according to claim 1, wherein the slit is provided in each of the protrusions.

8. A transfer instrument for transferring a medical sheet to a treatment site of a living body, the transfer instrument comprising:
an outer cylinder having a through hole that extends from a distal-end opening at a distal end of the outer cylinder to a proximal-end opening at a proximal end of the outer cylinder;
a carrier member that includes a shaft and a support portion, the shaft having a distal end portion and the support portion having a proximal-end support portion and a distal-end support portion, the proximal-end support portion terminating at a connection joint that is fixed to the distal end portion of the shaft so that movement of the shaft results in movement of the support portion;
the shaft being positionable in the through hole in the outer cylinder and being axially movable relative to the outer cylinder to move the support portion from a first position distal of the distal-end opening of the outer cylinder to a second position retracted into the through hole in the outer cylinder by axially moving the shaft in a proximal direction relative to the outer cylinder, and from the second position to the first position by moving the shaft in a distal direction relative to the outer cylinder;
the support portion including a support surface configured to support the medical sheet and an oppositely facing surface, the support portion including at least one slit that passes completely through the support portion so that the at least one slit opens to both the support surface of the support portion and the oppositely facing surface of the support portion so that moisture on the medical sheet supported on the support surface is able to flow through the at least one slit to be discharged to outside the support portion.

9. The transfer instrument according to claim 8, wherein the support portion includes a central region extending between the distal-end support portion of the support portion and the proximal-end support portion of the support portion, the plurality of slits including plural slits positioned on one side of the central part and plural slits positioned on an opposite side of the central part.

10. The transfer instrument according to claim 8, wherein the at least one slit includes a plurality of slits spaced apart from one another and located along opposites sides of the support portion, the opposite sides of the support portion being spaced apart from one another in a width-wise extent of the support portion.

11. The transfer instrument according to claim 8, wherein the support portion includes a bent portion that is bent, the at least one slit being located at the bent portion.

12. The transfer instrument according to claim 11, further comprising a protrusion that is connected to the bent portion and that projects away from the support surface.

13. The transfer instrument according to claim 8, wherein the support portion has a lengthwise extent between the distal-end support portion and the proximal-end support portion, the support portion having a widthwise extent perpendicular to the lengthwise extent, the support portion including two protrusions that project away from the support surface and that are spaced apart from one another in the widthwise extent.

14. The transfer instrument according to claim 8, wherein the proximal-end support portion is tapered in a narrowing manner towards the connection joint so that when the shaft is axially moved in the proximal direction relative to the outer cylinder the tapered proximal-end support portion contacts the distal-end opening at the distal end of the outer cylinder to cause the proximal-end support portion to curl along a circumferential direction of the outer cylinder.

15. The transfer instrument according to claim 8, wherein the support portion includes two bent portions that are each bent relative to an immediately adjacent part of the support portion, the two bent portions being spaced apart from one another and being located at opposite sides of the support portion, each bent portion extending in a respective extending direction, the at least one slit including two slits, each of the slits being positioned at a respective one of the bent portions and extending in the extending direction of the respective bent portion.

16. The transfer instrument according to claim 8, wherein the at least one slit includes a plurality of first cutouts spaced apart from one another and linearly arranged along one side of the support portion and a plurality of second cutouts spaced apart from one another and linearly arranged along an opposite side of the support portion.

17. The transfer instrument according to claim 8, wherein the shaft extends in an extending direction, the support portion having a width in a direction perpendicular to the extending direction of the shaft, the width of the support portion being greater than an inner circumferential length of the outer cylinder.

18. A method comprising:
moving a support portion toward a dish on which is supported a medical sheet, the support portion being fixed to a distal end portion of a shaft and the shaft being positioned in an outer cylinder so that a proximal end of the shaft extends proximally beyond a proximal end of the outer cylinder and the support portion extends distally beyond a distal end of the outer cylinder, the support portion including a support surface and an oppositely facing back surface, the support portion including at least one slit that passes completely through the support portion so that the at least one slit opens to both the support surface of the support portion and the back surface of the support portion;
positioning the medical sheet on the support surface of the support portion so that the medical sheet is supported on the support portion while the support surface of the support portion faces the medical sheet;
allowing moisture contained in the medical sheet to flow along the support surface and be discharged from the support surface toward the back surface of the support portion through the at least one slit; and
axially moving the shaft and the support portion in a proximal direction relative to the outer cylinder to retract the support portion into the outer cylinder while the medical sheet is supported on the support portion.

19. The method according to claim 18, wherein the support portion is deformed into a curved shape as the support portion is retracted into the outer cylinder, the shaft extending in an extending direction and the support portion having sides spaced apart from one another in a width-wise direction of the support portion, the width-wise direction of the support portion being orthogonal to the extending direction of the shaft, the at least one slit including a plurality of spaced apart cutouts positioned at both of the sides of the support portion, the allowing of the moisture contained in the medical sheet to flow along the support portion and to be discharged through the at least one slit including allowing of the moisture contained in the medical sheet to flow along the support portion and to be discharged through the cutouts on both sides of the support portion.

20. The method according to claim 19, wherein the support portion has sides spaced apart from one another in a width-wise extent of the support portion, and further comprising both of the sides of the support portion protruding toward the support surface after the support portion is retracted into the outer cylinder.
